(19) United States
(12) Patent Application Publication
(10) Pub. No.: US 2005/0255105 A1
(43) Pub. Date: Nov. 17, 2005

(54) Severe myelin deficits induced by self-reactive gamma delta T cells

(75) Inventors: Yueh-Hsiu Chien, Atherton, CA (US); Jung-Hua Yeh, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/088,159

(22) Filed: Mar. 22, 2005

Related U.S. Application Data
(60) Provisional application No. 60/555,740, filed on Mar. 22, 2004.

(51) Int. Cl. G01N 33/53; G01N 33/567; A61K 39/395

(52) U.S. Cl. 424/143.1; 435/7.2

(57) ABSTRACT

Recognition of ligands on myelinated axons by specific γδ T cells can induce demyelination. Among the ligands of interest are MHC molecules, including non-classical class I MHC molecules, which are shown to be expressed in cells of the oligodendrocyte lineage. These events provide a basis for initiating events of inflammatory demyelinating diseases.

Multiple sclerosis (MS) is an idiopathic inflammatory demyelinating disease of the CNS. Patients with MS commonly present with an individual mix of neuropsychological dysfunction, which tends to progress over years to decades. Inflammation and demyelination in the central nervous system (CNS) is a central feature found in patients with multiple sclerosis (MS). This disease has long been considered to be an autoimmune destruction mediated by immune cells against various components of the myelin sheath, but the responsiveness of T cells to myelin antigens has not always been found to associate with disease onset, or disease progression. For example, see Hemmer et al. (2002) Nat. Rev. Neurosci. 3, 291-301; and Keegan & Noseworthy (2002) Annu. Rev. Med. 53, 285-302.

Histopathological studies of MS lesions show demyelination and inflammation involving lymphocytes and activated macrophages/microglia in the myelinated areas of the brain and the spinal cord (Lassmann et al. (1998) J. Neuroimmunol. 86, 213-217). It is commonly accepted that αβ T cell and B cell responses specific for various components of the myelin sheaths contribute to the etiology of MS. The role of these autoimmune responses in disease have been largely established through the analysis of the animal model of experimental autoimmune encephalomyelitis (EAE) where inflammation and demyelination in the CNS can be induced by the injection of Freund's adjuvant together with CNS tissues or myelin components. Despite these advances, the primary event paving the way to the manifestation of the disease has remained elusive.

It has been shown that, unlike αβ T cell receptors, which recognize processed peptides in association with the MHC molecules, γδ T cell receptors can recognize antigens directly without antigen processing or presentation requirements (Schild et al. (1994) Cell 76:29-37). These receptors have been shown to interact with classical and non-classical MHC antigens, as well as non-protein ligands (for example, see Chien et al. (1996) Annu Rev Immunol. 14:511-32). Two well characterized ligands for γδ T cells are the non-classical MHC class I molecule T10 and the closely related T22. These ligands are recognized by a sizable population (˜0.2-2%) of γδ T cells in normal, unimmunized mice (Crowley et al. (2000) Science 287, 314-316). Recognition of these proteins by gamma delta T cell clones involves neither peptides bound to these proteins nor peptides derived from them (Crowley et al. (2000) Science 287, 314-316). Lipopolysaccharide (LPS)- or concanavalin A (conA)-activated splenocytes stimulate G8 and KN6 better than resting cells, and this activation of lymphocytes results in increased expression of T10 and/or T22 on the cell surface. In addition, T10/T22 mRNA has also been detected in the neocortical layers of the mature CNS (Crowley et al. (2000) Science 287, 314-316).

Gamma delta and alpha beta T cells appear to recognize antigens differently, although gamma delta T cells share many cell surface proteins with alpha beta T cells and are able to secrete lymphokines and express cytolytic activities in response to antigenic stimulation. Gamma delta T cells may mediate cellular immune functions without a requirement for antigen processing, by direct recognition of antigen without a requirement for antigen degradation or specialized antigen-presenting cells. The role of these cells in immune response pathways is of great interest for research and clinical purposes.

### SUMMARY OF THE INVENTION

Methods are provided for the diagnosis, prognosis and modulation of inflammatory demyelinating disease by interfering with the interaction between γδ T cells and cellular ligands, including non-classical and classical MHC molecules expressed in the central nervous system. Of interest are ligands of the γδ T cell receptor, which may include, without limitation, non-classical Class I MHC, Class II MHC, non-protein ligands, and the like.

In one embodiment of the invention, an animal model that utilizes the interaction between specific γδ T cell receptors and ligands in the central nervous system is provided for the development of therapeutic agents and methods of treatment for inflammatory demyelinating disease.

In another embodiment of the invention, the interaction between the γδ T cell receptor and ligands is used for screening agents therapeutic in the treatment of inflammatory demyelinating disease, according to an ability to interfere with the interaction.

In another embodiment, the detection of γδ T cells, particularly those that interact with antigens present on cells of the central nervous system, including cells in the oligodendrocyte lineage, peripheral blood monocytes/microglial that contribute to chronic neuroinflammatory lesions within the brain by entry across the blood brain barrier; etc., is an early diagnostic indicator of initiation or predisposition to multiple sclerosis.

Non-classical class I MHC molecules are shown to be expressed in cells of the oligodendrocyte lineage. Recognition of these proteins on myelinated axons by specific γδ T cells can induce demyelination. These events provide a basis for initiating events of inflammatory demyelinating diseases.

### BRIEF DESCRIPTION OF THE DRAWINGS

FIGS. 1A-1D. Cells in the oligodendrocyte lineage express T10 and stimulate G8 γδ T cells specific for T10. (1A) RT-PCR analysis of Tb10, and β-actin gene expression in spleen, liver, brain and spinal cord (S. C.) of two-month old BALB/c mice, dissociated spinal cord and DRG explants (D.) from E14 BALB/c embryos, freshly purified OPCs from P9 BALB/c mouse and in vitro differentiated oligodendrocytes. (1B) 7H9 antibody (specific for T10) staining of in vitro differentiated oligodendrocytes. Cells were fixed in 2% PFA on ice and stained with biotinylated 7H9 and strepavidin-PE. Scale bar, 7.5 μm. (1C) The Ca⁺⁺ response curve of G8 blasts in contact with in vitro differentiated oligodendrocytes. The images were taken every 20 seconds after adding G8 cells into. (1 D) Naïve G8 γδ T cells up-regulated CD69 after 48 hours co-cultured with spinal cord explants (red line). The control G8 γδ T cells, which were cultured in conditional media for 48 hours, are shown in gray line.

FIGS. 2A-2B. G8 γδ T cell blasts induce demyelination in myelinated cultures. (2A) Media alone (a, e) or γδ T cell blasts (1×10⁶) generated from BALB/c (b, 0, G8 (c, g), and G8/Rag^{-/-} (d, h) mice, were incubated with the dissociated spinal cord explants (a-d) and myelinated DRG explants (e-h) for 24 hours, fixed, and labeled with MBP specific antibody. The smooth, compact myelin sheath that was present in myelinated culture (a, e) was fragmented and bubbling (as indicated by arrowheads) in the (c, g) G8 and (d, h) G8/Rag^{-/-} cocultures. Data shown are representative of at least five independent experiments. (2B) Monomer blocking was performed by pre-incubating 1×10⁶ G8/Rag^{-/-} γδ T cell blasts with T22-β2M monomer (200 μg/ml) for 1 hour at room temperature before adding them to myelinated DRG explants. The monomers were present in the cocultures during the entire incubation period. The result shown is representative of four samples of two independent experiments.

FIG. 3. CFA injection induces G8 γδ T cell activation in the periphery. G8 γδ T cells were purified from the peripheral blood of IFA and CFA-injected G8 mice. The γδ T cells from IFA-injected G8 mice on Day 11 (dot line) and from CFA-injected G8 mice on day 4 (sold line) and day 11 (bold line) were stained with anti-PD-1 mAb. Data shown are representative of at least five mice.

FIG. 4A-H. G8 mice show the weakness in the limbs after CFA injection. (a) Time course showing the percentage of the mice exhibiting the weakness in the limbs from IFA (open triangle) and CFA (open square)-injected G8 mice and CFA-injected BALB/c mice (open circle). Data are representative of two independent experiments covering forty-six 6-week-old mice. (b-g) Increased number of γδ T cells, macrophages/microglia in the spinal cord from CFA-injected G8 mice. Serial frozen sections of the spinal cord stained for γδ T cells (b, c) or macrophages/microglia (d-g). The infiltration of γδ T cells was detected in CFA-injected G8 mice (c), but not in CFA-injected BALB/c mice (b). The presence of macrophages and microglia was also found only in CFA injected G8 mice (e, g), as compared to the CFA-injected BALB/c mice (d, f). Cumulative results are representative from twelve mice over at least eight independent experiments. (h) Splenic γδ T cells from IFA-(left) and CFA-(right) injected BALB/c mice were enriched and stained with T22-tetramers on day 11. Increased tetramer-positive cells were demonstrated in four different experiments covering eight mice.

FIG. 5. Characterization of G8/Shakie's Mbp gene. Analysis of G8/Shakie's Mbp gene structure. The lower part of the panel presents the result of the genomic Southern blot, for three restriction enzymes, with the DNA of wild type (+/+), G8/Shakie (s/s) and heterozygous (s/+) mice. The hybridization was carried out with a partial Mbp cDNA probe (see below). The scale for the size of the fragments is indicated on the left (in kb). The fragments unique (filled arrows) or absent (open arrow) in G8/Shakie's Mbp gene are presented on the right. The upper part of the panel summarizes the structure of Mbp in wild type mice (wt) and the deduced structure in G8/Shakie mice (shakie). The restriction sites are represented (S, SacI; X, XmnI; P, PstI) as well as the fragments generated that hybridized with the probe and their sizes on the right. Exons are numbered E1-7. The deleted segment in G8/Shakie is indicated by dotted lines. Open boxes represent the untranslated exons. The hybridization regions of the cDNA probe are represented below the map.

FIG. 6A-B. Expression of G8/Shakie's truncated Mbp gene. (A) Total RNA from brains of wild type (+/+), G8/Shakie (s/s) and heterozygous (s/+) mice were analyzed by northern blot. Three different ages of G8/Shakie were investigated (from left to right: 3-week, 2-month, 3-month). Arrowheads indicate the ribosomal RNA and their sizes. (B) Terminal exon in shakie mutant. The nucleic acid sequence of Mbp terminal exon 7 in wild-type (wt) and Shakie (shakie) mutant, as well as the corresponding amino acids sequence are indicated. The amino acids in bold represent two out of the five arginine residues. In Shakie, one arginine is replaced by an aspartic acid. * indicates the stop codon.

FIG. 7. Reduced lifespan of G8/Shakie (BALB/c/G8^{tg}-Mbp_{sha/sha}) compared to Shakie (BALB/c-Mbp^{sha/sha}). We recorded the number of days separating the birth from the natural death for each individual (G8/Shakie and Shakie). Each spot (·) represents one mouse, and the average for each group is indicated (−). Average lifespan of G8/Shakie is significantly reduced compared to Shakie (P<0.01, Student t-test). Similarly, Shakie displays an increased maximum lifespan comparing to G8/Shakie (G8/Shakie maximum lifespan: 93 days; Shakie minimum lifespan: 94 days).

FIG. 8A-B. MBP protein expression and amount of myelin in G8/Shakie and Shakie. (A) Western Blot analysis of the expression of the truncated MBP proteins in sha mutant. Total protein extract from the brains of BALB/c/G8^{tg}-Mbp^{sha/+} (sha/+), BALB/c/G8^{tg}-Mbp^{sha/sha} (G8/Sha) and BALB/c-Mbp^{sha/sha} (Sha) were used for Western Blot analysis. The molecular weights of the four MBP isoforms are shown on the left. (B) Electron micrographs of the optic nerve. The genotype of the mice from which the optic nerve were isolated is indicated above each photo. Scare bar, 2 μm; Ax, Axon.

FIG. 9. Normal cell numbers of NG2- and CC1-positive cells in the CNS of G8/Shakie. (A-C) Frozen sections of the brain were stained with anti-NG2 antibody (NG2, Chemicon) to identify OPCs in the white matter regions. The NG2-positive cells are similarly present in the brain from BALB/c (A), G8 (B) and G8/Shakie (C) mice. Scale bar, 50 μm. (D-F) Oligodendrocytes can be visualized by the CC-1 (Oncogene) staining in the optic nerves from BALB/c (D), G8 (E) and G8/Shakie (F) mice. Scale bar, 14 μm.

FIG. 10A-F. Accumulation of γδ T-cells in the white matter region of G8/Shakie's spinal cord. Serial frozen sections of the spinal cord were stained for macrophage/microglia (A-C, Mac-1-positive) or γδ T-cells (D-F, GL-3-positive). The presence of macrophage/microglia was detected in both Shakie and G8/Shakie (B,C). The massive accumulation of γδ T-cells was only found in G8/Shakie (F). Arrow indicates the blood vessel of the spinal cord.

### DETAILED DESCRIPTION OF THE EMBODIMENTS

Recognition of ligands on cells of the central nervous system by γδ T cells is shown to induce demyelination. Ligands of interest include, without limitation, non-classical class I MHC molecules, which are shown to be expressed in cells of the oligodendrocyte lineage; class II MHC, which are expressed on monocytes/microglial cells; and the like. These events can provide the basis for initiation of inflammatory demyelinating diseases, which diseases may include multiple sclerosis; EAE; optic neuritis; acute transverse myelitis; acute disseminated encephalitis; etc. The interaction between the γδ T cell receptor and ligands is useful in screening assays, where targeted disruption of the interaction is indicative that a compound may have activity in the prevention and treatment of inflammatory demyelinating disease. Compounds that interfere with this interaction find therapeutic use in the treatment of these diseases. The detection of specific γδ T cells in the central nervous system may be indicative of the early stages of a demyelinating disease, and find use as a diagnostic of initiation or predisposition to diseases such as MS. The presence of gamma delta T cells are also shown to exacerbate CNS pathology in neuroinflammatory conditions.

In one embodiment of the invention, an animal model that utilizes the interaction between specific γδ T cell receptors and ligands in the central nervous system is provided. These animals provide a useful model for the specific pathogenic requirements of inflammatory demyelinating disease, particularly in the initiation of the disease. By providing a model for the human disease, potential therapeutics can be evaluated in the animal model for safety and efficacy prior to clinical trials. In addition to screening candidate pharmaceutical agents, the subject animals are useful in determining the role of “triggering” agents in development of disease, the role of specific immune cell subsets and cytokines, and the role of specific antigens in activation; maintenance of inflammatory state; etc.

The animal models comprise a transgene encoding a γδ T cell receptor specific for a ligand of interest, e.g. an MHC class lb molecule, particularly where the transgene is specific for a class lb molecule of the T region, which may include one or more of T10 and T22. T cells expressing the transgene are activated, e.g. by the administration of adjuvant. The activating agent is preferably administered in the absence of exogenous myelin. After activation, the animals show a weakness of the limbs, associated with the presence of γδ T cells in the spinal cord, and increased numbers of macrophages and microglia. The γδ T cell infiltration can lead to the activation and accumulation of macrophages/microglia to initiate the immunological pathology in the CNS.

Another animal model provided herein comprises a single locus recessive genetic defect in the myelin basic protein gene. Among the amino acids replaced are one, two or more of the five arginine residues that are important for the attachment of MBP to the myelin membrane, resulting in a reduction in cationicity of the MBP proteins. This mouse model (Shakie) spontaneously develop marked tremors of the hindquarters at weaning. The amplitudes of these tremors increase as the mice move. The primary defect in these mice is a massive demyelination of the CNS. Almost all the axons of the optic nerve are naked in G8/Shakie mice that expressed the G8 γδ TCR transgenes in the shakie background. The presence of T10/T22 specific γδ T cells exacerbate CNS pathology.

### Non-Classical MHC

MHC class-I molecules are divided into several classes based on structural and functional properties. Class-Ib molecules, also termed the nonclassical class-I molecules, are structurally related to class-Ia molecules, but they display considerably less polymorphism, stimulate weak alloresponses, have tissue expression patterns ranging from cell-type-specific to ubiquitous, and appear to bind and present a much more limited range of ligands than the class-Ia molecules. These polypeptides ranging in molecular weight from 39-48 kDa and noncovalently associate with β2-microglobulin.

The genes encoding the class-Ia and class-Ib molecules are linked together as part of the major histocompatibility complex (MHC) found on human chromosome 6 and mouse chromosome 17. In the mouse, class Ib genes map to three defined regions, the Q, T, and M regions, adjacent to the H-2 complex. Of particular interest for the present invention is the T region, which can contain up to 18 class-I genes. Within this cluster are the genes that encode the Qa-1 molecule (gene T23), the serologically defined thymic leukemia antigens, TL, (genes T3 and T18) and the gene products of the T10 and T22 loci, which are recognized by γδ T-cell hybridomas.

The human HLA-linked human class-Ib molecules MICA and MICB are expressed in the intestinal epithelium in a stress-induced fashion and do not bind peptides. MICA and MICB are recognized by intestinal epithelial γδ T cells expressing the V1 receptor in either a TCR or NKG2D-dependent fashion. The MICA/B and T10/22 recognition systems may be cross-species counterparts.

### γδ T Cell Receptors

Each T cell receptor is a dimer consisting of one alpha and one beta chain or one delta and one gamma chain. In a single cell, the T cell receptor loci are rearranged and expressed in the order delta, gamma, beta, and alpha. The gamma locus includes V (variable), J (joining), and C (constant) segments.

About 5% of the T cells in the lymphoid organs of mice and humans express a TCR heterodimer consisting of the γ- and δ chains. Similar to αβ cells, γδ cells largely depend on the thymus for maturation and express T cell markers such as CD2, CD3, CD5, CD45, etc. The response of γδ cells to their ligand is similar to the response of αβ cells: γδ cells can kill target cells and can make lymphokines such as IL-2. Although γδ cells have been isolated that can recognize MHC molecules, these γδ cells recognize antigen differently from αβ cells.

Of main subsets in humans, Vγ9V/δ2 T cells predominate in the circulation. Another subset, defined by expression of Vδ1, is found in the intestinal epithelium, and may function as sentinels that respond to self antigens. The expression of a major histocompatibility complex (MHC) class I-related molecule, MICA, matches this localization. MICA and the closely related MICB are recognized by intestinal epithelial T cells expressing diverse Vδ1 TCRs. These interactions involve the α1α2 domains of MICA and MICB but are independent of antigen processing.

Several γδ cells have been isolated from mice and humans that recognize nonclassical MHC class I (class Ib) molecules, including Qa1; CD1; as well as Class II MHC; etc. The H-2T-encoded T10 and T22 proteins are ligands for γδ T cells. Animal models of the invention, as described below, may utilize one of the known T cell antigen receptors that recognize this molecule, e.g. the γ and δ chain G8; KN6; etc. (see Chien et al. (1996) Annu. Rev. Immunol. 14:511-532, herein incorporated by reference). The G8 γδ cell was isolated by allogeneic stimulation of spleen cells from an athymic mouse (Weintraub et al. (1994) J. I. 153:3051). Alternatively, the animal model may utilize a T cell receptor transgene obtained from a different species. For example, the human variable genes of the γ, δ regions have been characterized, and individual coding sequences may be selected for use (see Chien et al., supra.; Allison et al. (2002) Mol Immunol. 38(14):1051-61; among others).

### Demyelinating Inflammatory Disease Conditions

Multiple Sclerosis (MS) is the most common central nervous system (CNS) demyelinating disease, affecting 350,000 (0.1%) individuals in North America and 1.1 million worldwide. A widely used animal model of MS is experimental autoimmune encephalitis (EAE). Attacks of neurologic impairment occur in the early phase, which is characterized histologically by inflammatory lesions containing a predominance of CD4⁺ T cells, B cells and both MHC class II positive macrophages and microglia (a resident CNS antigen presenting cell). After multiple acute attacks a chronic “secondary progressive” phase with sustained neurologic impairment often ensues. This “irreversible” phase is characterized by neuronal loss and atrophy.

The pathologic hallmark of MS is multicentric, multiphasic CNS inflammation and demyelination. Recent studies have demonstrated that axonal transections occur during acute exacerbations; and axonal damage, as measured by magnetic resonance spectroscopy, was found to correlate with clinical disability.

Clinical symptoms of MS include sensory loss (paresthesias), motor (muscle cramping secondary to spasticity) and autonomic (bladder, bowel, sexual dysfunction) spinal cord symptoms; cerebellar symptoms (eg, Charcot triad of dysarthria, ataxia, tremor); fatigue and dizziness; impairment in information processing on neuropsychological testing; eye symptoms, including diplopia on lateral gaze; trigeminal neuralgia; and optic neuritis. The diagnosis of MS is based on a classic presentation (ie, optic neuritis, transverse myelitis, internuclear ophthalmoplegia, paresthesias) and on the identification of other neurologic abnormalities, which may be indicated by the patient history and exam. Typical findings on an MRI also help establish a diagnosis of MS.

About 70% of patients present with the more favorable relapsing-remitting (RR) type, which is characterized by acute exacerbations with full or partial remissions. For patients with RR, the FDA has approved the long-term use of beta-interferons and glatiramer acetate, which is a synthetic form of myelin basic protein (MBP). Both have demonstrated reductions of approximately 33% in both clinical disease activity and progression of MS lesions on MRI. The remaining patients present with chronic progressive MS, which is subdivided further into primary-progressive (PP), relapsing-progressive (RP), which is a pattern combining features of RR and RP and is intermediate in clinical severity, and secondary-progressive (SP), which many patients with RR progress to over time.

MS commonly is believed to result from an autoimmune process. What triggers the autoimmune process is not clear, but the nonrandom nature of its geographic distribution have suggested an isolated or additive environmental effect and/or inadvertent activation and dysregulation of CNS immune processes by a infection. Some authorities have suggested that human herpesvirus-6 (HHV-6) variant B group 2, while others implicate Chlamydia pneumonia.

Polygene inheritance accounts for a familial rate of 10-20%; and a monozygotic twin has a 30% risk of acquiring MS, suggesting a genetic predisposition to an environmental viral agent. Human leukocyte antigen (HLA) patterns of patients with MS tend to differ from those of the general population.

Treatment of progressive disease or prevention of relapses may involve use of interferon, cyclosporine, azathioprine, methotrexate, or other immunomodulatory agents. Dexamethasone is used in the treatment of acute transverse myelitis and acute disseminated encephalitis, for example at a high dose of 500 mg IV for 5 days. Dexamethasone is often prescribed for autoimmune diseases, e.g. in an adult at a dose of 10 mg IV every 6 hours. A variety of medications may be provided for treatment of symptoms such as spasticity, trigeminal neuralgia, depression/fatigue, etc.

### Compound Screening

Compound screening may be performed using a cellular interaction model, a genetically altered cell or animal, or purified ligands that provide for an interaction between a γδ T cell receptor and ligand. Cells of interest for screening include microglia; oligodendrocytes and other cells in the oligodendrocyte lineage, including progenitor cells thereof. These cells may express native ligands, e.g. class II; class Ib molecules, or may be genetically altered to express an exogenous ligand. T cells expressing γδ T cell receptors, including receptors specific for class Ib molecules, are also of interest for screening. One can identify ligands or substrates that bind to, and/or modulate the interaction of receptor and ligand.

For in vitro screening methods, the ligands, which may include, without limitation, class II, non-classical class I such as MHC class Ib; etc., may be provided in a soluble form, and may further be multimerized to enhance binding to the T cell receptor. Methods of such multimerization are known in the art, although the complexes typically used for αβ T cell receptor binding include an antigenic peptide component, which may not be necessary for the interaction with γδ TCR. The MHC chains may be multimerized through fusion to a multivalent protein, e.g. immunoglobulin, or by binding the monomers to a multivalent entity through specific attachment sites. A multimer may also be formed by chemical cross-linking. The attachment site for binding to a multivalent entity may be naturally occurring, or may be introduced through genetic engineering. The site can be a specific binding pair member or one that is modified to provide a specific binding pair member, where the complementary pair has a multiplicity of specific binding sites. Binding to the complementary binding member can be a chemical reaction, epitope-receptor binding or hapten-receptor binding where a hapten is linked to the subunit chain. One of the subunits can be fused to an amino acid sequence providing a recognition site for a modifying enzyme, for example BirA, various glycosylases, farnesyl protein transferase, protein kinases and the like. The subunit may be reacted with the modifying enzyme at any convenient time, usually after formation of the monomer. The group introduced by the modifying enzyme, e.g. biotin, sugar, phosphate, farnesyl, etc. provides a complementary binding pair member, or a unique site for further modification, such as chemical cross-linking, biotinylation, etc. that will provide a complementary binding pair member.

For screening methods, it may not be necessary, or even desirable, to use the complete proteins for studying interactions. Soluble forms of the components, which lack the transmembrane domains, are of interest for in vitro screening. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site, or to minimize misfolding by substitution or deletion of one or more cysteine residues that are not necessary for function. Variants can be designed so as to retain or have enhanced biological activity of a particular region of the protein (e.g., a functional domain, transmembrane domain, etc.) Variants also include fragments of the polypeptides, particularly biologically active fragments and/or fragments corresponding to functional domains, which are sufficient for specific binding interactions. Fragments of interest will typically be at least about 10 aa to at least about 15 aa in length, usually at least about 50 aa in length, and can be as long as 300 aa in length or longer.

Transgenic animals or cells derived therefrom are also used in compound screening. Transgenic animals may be made through homologous recombination, particularly where the transgene encodes a γδ T cell receptor. Vectors for stable integration include plasmids, retroviruses and other animal viruses, YACs, and the like. A series of small deletions and/or substitutions may be made in the coding sequence to determine the role of different exons in signal transduction, etc. Other constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the ligand or TCR in cells or tissues where they are not normally expressed, or at abnormal times of development. By providing expression of the target protein in cells in which it is not normally produced, one can induce changes in cell behavior. Animal models of interest may also comprise γδ T cells that are activated as a result of administering an adjuvant, e.g. Freund's Complete Adjuvant.

Compound screening identifies agents that modulate function of the γδ T cells with respect to cells of the central nervous system, e.g. microglial; cells of the oligodendrocyte lineage; etc. Of particular interest are screening assays for agents that have a low toxicity for human cells. A wide variety of assays may be used for this purpose, including labeled in vitro protein-protein binding assays, functional assays for T cell activation, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering the physiological function of a γδ T cell, preferably altering in a targeted, i.e. antigen specific, manner. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of interfering with the binding between a γδ T cell receptor and ligand, as at least some of the compounds so identified are likely inhibitors. The binding assays usually involve contacting a cell or combination of cells or protein or combination of proteins with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89.

Certain screening methods involve screening for a compound that modulates the expression of a γδ T cell receptor gene. Such methods generally involve conducting cell-based assays in which test compounds are contacted with one or more cells expressing a γδ T cell receptor gene and then detecting a decrease in expression. Some assays are performed with T cells that express endogenous TCR genes.

The level of expression or activity can be compared to a baseline value. The baseline value can be a value for a control sample or a statistical value that is representative of expression levels for a control population. Expression levels can also be determined for cells that do not express a TCR gene, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to an animal that serves as a model for humans and then determining the initiation and/or progression of disease. The animal models utilized in validation studies generally are mammals. Specific examples of suitable animals include, but are not limited to, primates, mice, and rats.

Active test agents identified by the screening methods described herein that specifically inhibit the interaction between γδ T cell receptor and ligand; and/or inhibit the initiation and/or progression of inflammatory demyelinating disease can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

### Pharmaceutical Formulations

As used herein, the term “treating” is used to refer to both prevention of disease, and treatment of pre-existing conditions. In the treatment of ongoing disease, the treatment stabilizes or reduces the undesirable clinical symptoms of the patient. Such treatment is desirably performed prior to complete loss of function in the affected tissues. The subject therapy may be administered before, or during the symptomatic stage of the disease, and in some cases after the symptomatic stage of the disease.

Compounds this identified may be administered for preventing the initiation, or the progression of an inflammatory demyelinating disease. One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to the central nervous system is also an option. A BBB disrupting agent can be co-administered with the therapeutic or imaging compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration, injection into the cerebrospinal fluid, intracavity or direct injection in the demyelinating disease. Intrathecal administration maybe carried out through the use of an Ommaya reservoir, in accordance with known techniques. (F. Balis et al., Am J. Pediatr. Hematol. Oncol. 11, 74, 76 (1989).

The effective amount of a therapeutic composition to be given to a particular patient will depend on a variety of factors, several of which will be different from patient to patient. A competent clinician will be able to determine an effective amount of a therapeutic agent to administer to a patient to retard the harmful inflammation, or an effective amount of an imaging composition to administer to a patient to facilitate the diagnosis and visualization of demyelinated lesions. Dosage of the agent will depend on the treatment of the demyelinating disease, route of administration, the nature of the therapeutics, sensitivity of the disease to the therapeutics, etc. Utilizing LD₅₀ animal data, and other information, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Imaging moieties are typically less toxic than cytotoxic moieties and may be administered in higher doses in some embodiments. Utilizing ordinary skill, the competent clinician will be able to optimize the dosage of a particular therapeutic or imaging composition in the course of routine clinical trials.

Typically the dosage will be 0.001 to 100 milligrams of conjugate per kilogram subject body weight. Relatively large doses, in the range of 0.1 to 10 mg per kilogram of patient body weight, may used for imaging conjugates with a relatively non-toxic imaging moiety. The amount utilized will depend on the sensitivity of the imaging method, and the relative toxicity of the imaging moiety.

### Imaging

A specific binding partner of interest for diagnostic purposes, and in some instances for therapeutic purposes, is an antibody. For imaging purposes, the antibody may be specific for γδ T cells without being specific for a particular antigen receptor, e.g. a constant or invariant region of the receptor; a non-antigen receptor marker that differentiates γδ T cells from other cells, etc. In other embodiments, the antibody will specifically interfere with the interaction between a γδ T cell and a cell expressing a class Ib MHC; class II MHC; CD1; Qa1; etc. ligand. While the antibodies may be used without conjugation, in some embodiments the specific antibodies are conjugated to cytotoxic or imaging agents, which add functionality.

The antibodies can be coupled or conjugated to one or more therapeutic cytotoxic or imaging moieties. As used herein, “cytotoxic moiety” is a moiety that inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins, and derivatives thereof. “Imaging moiety” (I) is a moiety that can be utilized to increase contrast between a lesion, e.g. in the CNS, and the surrounding healthy tissue in a visualization technique (e.g., radiography, positron-emission tomography, magnetic resonance imaging, direct or indirect visual inspection). Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.). It will be appreciated by one of ordinary skill that some overlap exists between therapeutic and imaging moieties. For instance ²¹²Pb and ²¹²Bi are both useful radioisotopes for therapeutic compositions, but are also electron-dense, and thus provide contrast for X-ray radiographic imaging techniques, and can also be utilized in scintillation imaging techniques.

In general, therapeutic or imaging agents may be conjugated to the antibody moiety by any suitable technique, with appropriate consideration of the need for pharmacokinetic stability and reduced overall toxicity to the patient. A therapeutic agent may be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Two or more cytotoxic and/or imaging moieties may be conjugated to an antibody, where the conjugated moieties are the same or different. By poly-derivatizing the antibody, several cytotoxic strategies can be simultaneously implemented; an antibody may be made useful as a contrasting agent for several visualization techniques; or a therapeutic antibody may be labeled for tracking by a visualization technique. Immunoconjugates with more than one moiety may be prepared in a variety of ways. For example, more than one moiety may be coupled directly to an antibody molecule, or linkers which provide multiple sites for attachment (e.g., dendrimers) can be used. Alternatively, a carrier with the capacity to hold more than one cytotoxic or imaging moiety can be used.

Carriers and linkers specific for radionuclide agents (both for use as cytotoxic moieties or positron-emission imaging moieties) include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use in magnetic resonance imaging disease visualization methods, and for the chelation of heavy metal ions for use in radiographic visualization methods.

Preferred radionuclides for use as cytotoxic moieties are radionuclides that are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other β-radiation emitting nuclides, which have an effective range of several millimeters. ¹²³I, ¹²⁵I, ¹³¹I, or ²¹¹At may be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope may be utilized in the recited iodo-reagents. Radionuclides can be conjugated to antibody moieties by suitable chelation agents known to those of skill in the nuclear medicine arts.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions which may be utilized for x-ray radiography are described in U.S. Pat. No. 5,709,846, incorporated fully herein by reference. Such moieties may be conjugated to the antibody moiety through an acceptable chemical linker or chelation carrier. In addition, radionuclides which emit radiation capable of penetrating the skull may be useful for scintillation imaging techniques. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction.

Preferred magnetic resonance contrast moieties include chelates of chromium(III), manganese(II), iron(II), nickel(II), copper(II), praseodymium(II), neodymium(II), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the antibody moieties through a suitable chemical linker.

### Combination Therapies

In some cases, it may be preferred to use various combinations of therapeutic or imaging agents. Such combination treatments may be by administering blended therapeutic or imaging compositions, individually prepared as described above, and administering the blended therapeutic to the patient as described. The skilled administering physician will be able to take such factors as combined toxicity, and individual agent efficacy, into account when administering such combined agents. Additionally, those of skill in the art will be able to screen for potential cross-reaction with each other, in order to assure full efficacy of each agent. The agents of the invention may also be administered in combination with other therapies, e.g. beta-interferon, peptides, etc. known for the treatment of such diseases.

### Animal Models

Animals of the invention, as described above, may have a transgene of a T cell receptor specific for a class Ib protein, particularly a T10/T22 or homolog thereof. The T cells are typically activated to imitate the initiation of disease, e.g. by administration of adjuvant, which adjuvant may include bacteria or bacterial antigens, usually inactive bacteria. Unlike other models of demyelinating disease, the adjuvant is administered in the absence of exogenous components of the myelin sheath, such as myelin, myelin fragments, PLP, etc. Transgenic mammals of use as models for disease are usually small laboratory animals, including rodents such as mice, rats, etc. Other species of transgenic mammals have also been created and may find use, e.g. pigs, goats, dogs, etc. Generally, the host will be at least about four weeks old. For example, mice are often used at about 4 to 12 weeks of age. The mammalian host will be grown in conventional ways.

The presence of gamma delta T cells are also shown to exacerbate CNS pathology as exemplified by the increased severity of CNS pathology and shortened life span of the ‘Shakie’ mice on the G8 transgenic mice background versus ‘Shakie’ mice. Shakie, a spontaneous developed mutant mouse found in our mouse colony, has a single locus recessive genetic defect in the myelin basic protein gene. It has a 9 kb deletion, which removes exon 6 and the translated part of the terminal exon 7 of Mbp gene. One major form of the Mbp mRNA expressed in Shakie is similar to the mRNA encoding the 17.2 kDa isoform of MBP (lacking exon 6) in normal mice, but with a different 3′ end, containing 27 bp of the untranslated region of terminal exon 7. This results a replacement of the 14 amino acids encoded by exon 7 found in all the isoforms of MBP by 9 new amino acids. Among the amino acids replaced are two out of the five arginine residues that are important for the attachment of MBP to the myelin membrane. A reduction in cationicity of the MBP proteins has been thought to play an important role in the pathogenesis of demyelinating diseases. There has been reports showing high incidence of citrullinated MBP (MBP protein with conversion of the arginine residue to the uncharged citrulline) in chronic (45%) and fulminating (90%) MS patients as compared to healthy individuals (20%).

Shakie spontaneously develop marked tremors of the hindquarters at weaning. The amplitudes of these tremors increase as mice the moved. The primary defect in these mice does not appear to be a shortage of the myelinating oligodendrocytes. Instead, there is a massive demyelination of the CNS. Despite Shakie mice show a significant decreased myelin of the optic nerve when compared to that of the heterozygous mutant (sha/+) or the wild type mice, almost all the axons of the optic nerve are naked in G8/Shakie mice which expressed the G8 gd TCR transgenes in the shakie background.

In addition, G8 mice have a normal lifespan; Shakie have an average lifespan of 107 days, but that of G8/Shakie is only around 76 days of (p<0.01). Taken together, these results showed that the presence of T10/T22 specific gd T cells exacerbate CNS pathology.

While myelinated axons were absent, the numbers and appearance of OPCs (NG2-positive) and oligodendrocytes (CC1-positive) in the white matter area of the G8/Shakie's brain are comparable with that of Shakie, G8 as well as BALB/C mice. Thus, an interruption of OPC development is unlikely to be the cause of complete myelin deficiency in G8/Shakie. In addition macrophage/microglia (Mac-1-positive) are increased in the white matter region of the spinal cord in both Shakie and G8/Shakie. In contrast, a massive accumulation of γδ T-cells (GL-3-positive) is only found in G8/Shakie. This indicates that the infiltration of T10/T22 specific G8 γδ T-cells in the white matter may be responsible for the complete demyelination in the CNS of G8/Shakie mice.

To more fully characterize the disease, immunophenotypic analysis may be performed to detect a variety of relevant parameters. To characterize the types of cells present in lesions, immunohistochemical stains for various markers may be performed, including leukocyte markers including T cell markers for subsets such as PD-1; CD69; CD4, antigenic specificities; microglial cell markers such as Mac-1, F4/80; cytokines such as γ-IFN, IL-2, etc. The expression of additional adhesion molecules that are relevant to the pathophysiology of chronic inflammatory disease may include mononuclear cell infiltrate; T cells at lesions; and the expression in adjacent blood vessels of focal E-selectin, P-selectin, ICAM-1 and diffuse vascular cell adhesion molecule-1 (VCAM-1) expression.

The subject animals are useful for screening candidate therapeutic agents and treatment modalities. Through use of the subject animals or cells derived therefrom, one can identify ligands or substrates that affect the progression of inflammatory demyelinating disease. Drug screening protocols may include a panel of animals, for example a test compound or combination of test compounds, and negative and/or positive controls, where the positive controls may be known immunosuppressive agents. Such panels may be treated in parallel, or the results of a screening assay may be compared to a reference database.

Depending on the particular assay, whole animals may be used, or cells derived therefrom. Cells may be freshly isolated from an animal, or may be immortalized in culture. Candidate therapies may be novel, or modifications of existing treatment options. For screening assays that use whole animals, a candidate agent or treatment is applied to the subject animals. Typically, a group of animals is used as a negative, untreated or placebo-treated control, and a test group is treated with the candidate therapy. Generally a plurality of assays are run in parallel with different agent dose levels to obtain a differential response to the various dosages. The dosages and routes of administration are determined by the specific compound or treatment to be tested, and will depend on the specific formulation, stability of the candidate agent, response of the animal, etc.

The analysis may be directed towards determining effectiveness in prevention of disease induction, where the treatment is administered before induction of the disease, i.e. prior to injection of the T cells and/or pro-inflammatory cytokine. Alternatively, the analysis is directed toward regression of existing lesions, and the treatment is administered after initial onset of the disease, or establishment of moderate to severe disease. Frequently, treatment effective for prevention is also effective in regressing the disease.

In either case, after a period of time sufficient for the development or regression of the disease, the animals are assessed for impact of the treatment, by visual, histological, immunohistological, and other assays suitable for determining effectiveness of the treatment. The results may be expressed on a semi-quantitative or quantitative scale in order to provide a basis for statistical analysis of the results.

The term “agent” as used herein describes any molecule, e.g. protein or pharmaceutical, with the capability of affecting the severity of inflammatory demyelinating disease. An agent or treatment is administered to an animal of the invention, or to cells derived therefrom. Antibodies specific for cytokines, T cell receptor, polyclonal activating agents, T cell antigens, etc. are of interest.

### Antigen Specific Therapeutic Methods

The antigens or epitopes of the γδ T cell receptor and/or ligand, e.g. class Ib MHC; class I MHC; class II MHC, etc. can be utilized to develop and select antigen or epitope specific therapies, which comprise administration of an antigen or epitope specific therapeutic agent. Therapeutic methods include oral administration of specific-antigens, termed ‘oral tolerance’ (Annu Rev Immunol. 12:809-37); administration of native peptides (Science 258:1491-4; J Neurol Sci. 152:31-8); administration of altered peptide ligands (Nature 379:343-5); administration of whole proteins (Science 263:1139); administration of fusion-proteins or peptides; administration of other molecules, such as DNA or allergens including pollen, dust mites, cat salivary antigen (J. Rheumatology 28:257-65); administration of polynucleotide sequences encoding the targeted T cell receptor; and the like. For all of these therapies, the antigens administered (or encoded in DNA) for purposes of immune suppression may comprise all or a portion of the TCR; class Ib protein, etc. In one embodiment, one or more of the epitopes thus identified are administered, usually two or more, more usually three or more, and may comprise as many as ten or more different epitopes. Individual peptides or DNA encoding peptides may be administered. Alternatively, whole proteins, or DNA encoding all or substantially all of the antigenic protein may be administered. One or more, usually two or more, and as many as three of more different protein antigens may be thus administered.

In one embodiment, treatment comprises the induction of an antigen-specific, suppressive T-cell response by administration of a DNA expression cassette injected into host tissue, for example muscle or skin. The vector comprises a DNA sequence encoding at least a portion of a γδ TCR, etc. In response to this treatment, a suppressive response is evoked. T cell proliferation is thus inhibited.

The prevention of autoimmune disease involving the targeted TCR is accomplished by administration of the agent prior to development of overt disease. The treatment of ongoing disease, where the treatment stabilizes or improves the clinical symptoms of the patient, is also of particular interest.

A DNA expression cassette encoding all, substantially all, or a portion of a γδ TCR, encoding at least one complete epitope, usually as part of a vector, is introduced into tissue of the recipient. The gene, or minigene, is expressed in the tissue, and the encoded polypeptide acts to suppress the immune response. The DNA expression cassette will comprise most or all of the sequence encoding a γδ TCR fragment. The coding sequence may be truncated at the 5′ or 3′ terminus and may be a fragment of the complete polypeptide sequence.

The immune response may be enhanced by the inclusion of CpG sequences, as described by Krieg et al. (1998) Trends Microbiol. 6:23-27, and helper sequence, King et al. (1998) Nat. Med. 4:1281-1286. Biological effects of DNA motifs like unmethylated CpG dinucleotides, in particular base contexts (CpG-S motifs), may modulate innate immune responses when injected into animals.

The γδ TCR sequences are inserted into an appropriate expression cassette. The expression construct is prepared in conventional ways. The cassette will have the appropriate transcriptional and translational regulatory sequences for expression of the sequence in the recipient cells. The cassette will generally be a part of a vector, which contains a suitable origin of replication, and such genes encoding selectable markers as may be required for growth, amplification and manipulation of the vector, prior to its introduction into the recipient. Suitable vectors include plasmids, YACs, BACs, bacteriophage, retrovirus, adenovirus, and the like. Conveniently, the expression vector will be a plasmid. Prior to introduction into the recipient, the cassette may be isolated from vector sequences by cleavage, amplification, etc. as known in the art. For injection, the DNA may be supercoiled or linear, preferably supercoiled. The cassette may be maintained in the host cell for extended periods of time, or may be transient, generally transient. Stable maintenance is achieved by the inclusion of sequences that provide for integration and/or maintenance, e.g. retroviral vectors, EBV vectors and the like.

The expression cassette will generally employ an exogenous transcriptional initiation region, i.e. a promoter other than the promoter which is associated with the T cell receptor in the normally occurring chromosome. The promoter is functional in host cells, particularly host cells targeted by the cassette. The promoter may be introduced by recombinant methods in vitro, or as the result of homologous integration of the sequence by a suitable host cell. The promoter is operably linked to the coding sequence of the antigen to produce a translatable mRNA transcript. Expression vectors conveniently will have restriction sites located near the promoter sequence to facilitate the insertion of antigen sequences.

Expression cassettes are prepared comprising a transcription initiation region, which may be constitutive or inducible, the gene encoding the antigen sequence, and a transcriptional termination region. The expression cassettes may be introduced into a variety of vectors. Promoters of interest may be inducible or constitutive, usually constitutive, and will provide for transcription in the recipient cells. The promoter may be active only in the recipient cell type, or may be broadly active in many different cell types. Many strong promoters for mammalian cells are known in the art, including the β-actin promoter, SV40 early and late promoters, immunoglobulin promoter, human cytomegalovirus promoter, retroviral LTRs, etc. The promoters may or may not be associated with enhancers, where the enhancers may be naturally associated with the particular promoter or associated with a different promoter.

A termination region is provided 3′ to the coding region, where the termination region may be naturally associated with the variable region domain or may be derived from a different source. A wide variety of termination regions may be employed without adversely affecting expression.

The DNA vectors are suspended in a physiologically acceptable buffer, generally an aqueous solution e.g. normal saline, phosphate buffered saline, water, etc. Stabilizing agents, wetting and emulsifying agents, salts for varying the osmotic pressure or buffers for securing an adequate pH value, and skin penetration enhancers can be used as auxiliary agents. The DNA will usually be present at a concentration of at least about 1 ng/ml and not more than about 10 mg/ml, usually at about from 100 μg to 1 mg/ml.

The DNA tolerizing therapeutic may be fractionated into two or more doses, of at least about 1 μg, more usually at least about 100 μg, and preferably at least about 1 mg per dose, administered from about 4 days to one week apart. In some embodiments of the invention, the individual is subject to a series of vaccinations to produce a full, broad immune response. According to this method, at least two and preferably four injections are given over a period of time. The period of time between injections may include from 24 hours apart to two weeks or longer between injections, preferably one week apart. Alternatively, at least two and up to four separate injections are given simultaneously at different parts of the body.

The DNA therapeutic is injected into muscle or other tissue subcutaneously, intradermally, intravenously, orally or directly into the spinal or synovial fluid. Of particular interest is injection into skeletal muscle. The genetic therapeutic may be administered directly into the individual to be immunized or ex vivo into removed cells of the individual which are reimplanted after administration. By either route, the genetic material is introduced into cells which are present in the body of the individual. Alternatively, the genetic therapeutic may be introduced by various means into cells that are removed from the individual. Such means include, for example, transfection, electroporation and microprojectile bombardment. After the genetic construct is taken up by the cells, they are reimplanted into the individual. Otherwise non-immunogenic cells that have genetic constructs incorporated therein can betaken from one individual and implanted into another.

Bupivacaine or compounds having a functional similarity may be administered prior to or contemporaneously with the vaccine. Bupivacaine is a homologue of mepivacaine and related to lidocaine. It renders muscle tissue voltage sensitive to sodium challenge and effects ion concentration within the cells. In addition to bupivacaine, mepivacaine, lidocaine and other similarly acting compounds, other contemplated cell stimulating agents include lectins, growth factors, cytokines and lymphokines such as platelet derived growth factor (PDGF), gCSF, gMCSF, epidermal growth factor (EGF) and IL-4.

### Diagnostic and Prognostic Methods

The presence of γδ T cells at the initiation of inflammatory demyelinating disease indicates that these can serve as markers for diagnosis, for imaging, as well as for therapeutic applications. In general, such diagnostic methods involve detecting an elevated level of γδ T cells in the cerebral spinal fluid (CSF), or neural tissues an individual, or a sample therefrom, particularly γδ T cells specific for a ligand of interest, e.g. class Ib MHC; class I MHC; class II MHC, etc. A variety of different assays can be utilized to detect an increase in such cells. More specifically, the diagnostic and prognostic methods disclosed herein involve obtaining a sample from an individual and determining at least qualitatively, and preferably quantitatively, the level of γδ T cells in the sample. Usually this determined value or test value is compared against some type of reference or baseline value.

Binding members such as antibodies that are specific for these cells are used to screen patient samples for increased numbers of cells. Samples can be obtained from a variety of sources. Samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from spinal fluid, or biopsy samples. Also included in the term are derivatives and fractions of such cells and fluids. Samples can also be derived from in vitro cell cultures, including the growth medium, recombinant cells and cell components. Diagnostic samples are collected from an individual that has, or is suspected to have, an inflammatory demyelinating disease. The presence of specific markers is useful in identifying and staging the disease.

Detection may utilize staining of cells or histological sections, performed in accordance with conventional methods, using antibodies or other specific binding members. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and γδ T cell receptor in a lysate or other sample. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding. After incubation, the insoluble support is generally washed of non-bound components. After washing, a solution containing a second antibody is applied. The antibody will bind to one of the proteins of interest with sufficient specificity such that it can be distinguished from other components present. The second antibodies may be labeled to facilitate direct, or indirect quantification of binding. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

In some embodiments, the methods are adapted for imaging use in vivo, e.g., to locate or identify sites where γδ T cells or demyelinated lesions are present. In these embodiments, a detectably-labeled moiety, e.g., an antibody is administered to an individual (e.g., by injection), and labeled cells are located using standard imaging techniques, including, but not limited to, magnetic resonance imaging, computed tomography scanning, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized. A currently used method for labeling with ^{99m}Tc is the reduction of pertechnetate ion in the presence of a chelating precursor to form the labile ^{99m}Tc-precursor complex, which, in turn, reacts with the metal binding group of a bifunctionally modified chemotactic peptide to form a ^{99m}Tc-chemotactic peptide conjugate.

The detectably labeled specific antibody is used in conjunction with imaging techniques, in order to analyze the expression of the target. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (γ-ray) emissions of the specific isotopes employed and the sensitivity and sophistication of the instruments used to detect them, the two-dimensional distribution of radioactivity may be inferred from outside of the body.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the protein” includes reference to one or more proteins and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Recognition of Non-Classical Class I MHC Molecules on Neuronal Myelinating Cells by γδ T Cells

Mice carrying a spontaneous mutation in the myelin basic protein gene and expressing a transgenic G8 γδ TCR specific for T10/T22 have been found to show a massive accumulation of cells, including G8 γδ T cells and macrophages/microglial, in the white matter of the spinal cord. This pathology is suggestive that T10/T22 is expressed by myelin forming cells in the white matter and their recognition contributes to the retention of G8 γδ T cells in the CNS.

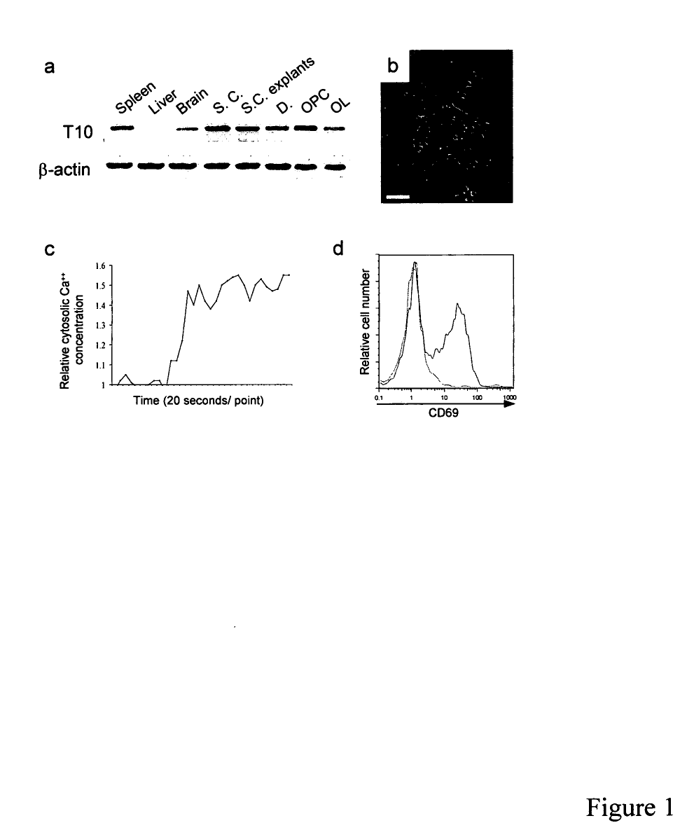

Indeed, as shown by RT-PCR, T10/T22 mRNA can be found in freshly isolated spinal cord, the oligodendrocyte precursor cells (OPCs) from 9 days postnatal (P9) BALB/c mice and oligodendrocytes differentiated 7 days from OPCs in vitro (FIG. 1a). More significantly, the expression of T10/T22 on the in vitro differentiated oligodendrocytes can be detected with a T10/T22 specific antibody 7H9 (FIG. 1b). This is the first demonstration of non-classical class I MHC expression on myelin forming cells in the CNS.

To test whether G8 cells can recognize oligodendrocyte precursor/oligodendrocytes, we first prepared OPCs from the brains of P9 BALB/c mice and tested whether G8 γδ T cells can respond to these cells by fluxing calcium. As shown in FIG. 1c, a steep rise and sustained elevation of intracellular calcium concentration can be observed in G8 γδ T cells that contact the OPCs. The calcium flux patterns are similar to those observed in αβ T cells contacting agonist peptides presented by antigen presenting cells (Wulfing et al. (2002) Nat. Immunol. 3, 42-47).

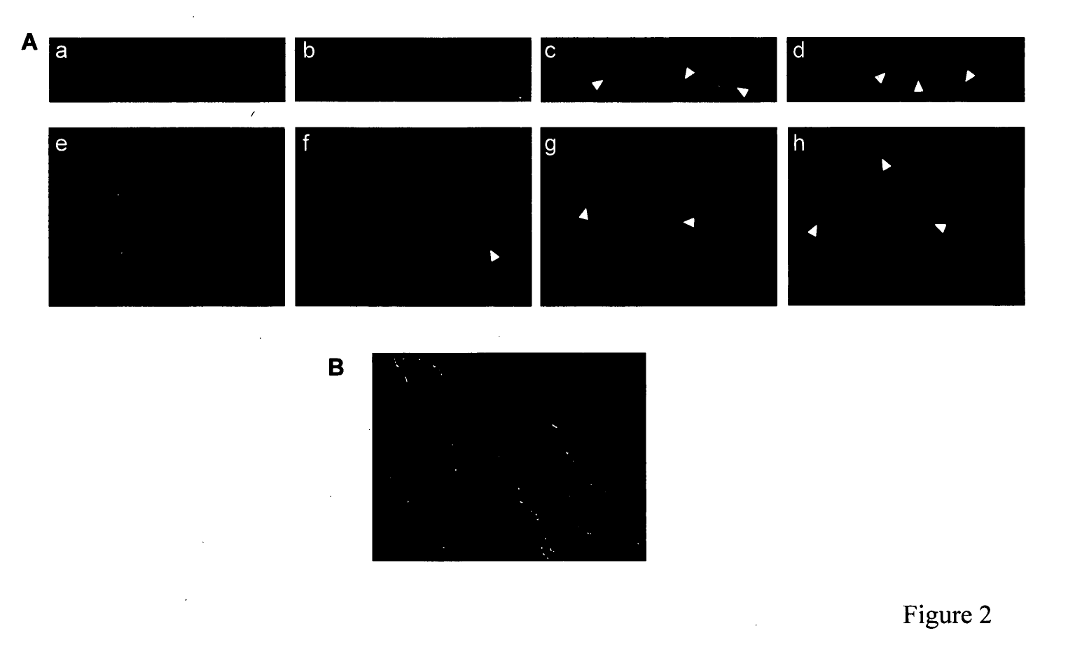

To further investigate the consequences of G8 γδ T cells-oligodendrocyte precursor/oligodendrocytes interaction, it was tested whether G8 γδ T cells can survive in the in vitro myelinated cultures of dissociated spinal cord or dorsal root ganglia (DRG) explants. These cultures were carried out in minimal tissue culture media with neural-growth factor (NGF) as the only supplement, without any cytokines or specific antibodies. Surprisingly, it was found that naïve G8 γδ T cell were activated after 48 hr co-incubation with the myelinating cocultures of dissociated spinal cord or DRG explants (FIG. 1d). Furthermore, when G8 γδ T cell blasts were added to myelinated cultures of dissociated spinal cord or DRG explants, extensive and rapid demyelination was observed (FIG. 2B, 2F).

The demyelination appears to be initiated due to the recognition of T10/T22 on the myelinating cells by G8 γδ T cells. G8/Rag^{-/-} γδ T cell blasts which only express the transgenic G8 γδ TCR were even more effective in eliminating myelin internodes (FIG. 2C, 2G). In contrast, γδ T cell blasts from BALB/c mice, which express diverse γδ TCRs had only a slight effect (FIG. 2D, 2H). In addition, the demyelination effect was effectively blocked when the G8/Rag^{-/-} γδ T cell antigen recognition was inhibited by pre-incubating the cells with monomeric T22-β2M and including the monomeric T22-β2M in the incubation with the explant culture (FIG. 21). Taken together, these results demonstrate that a major population of γδ T cells can recognize specific neuronal cells and induce pathology.

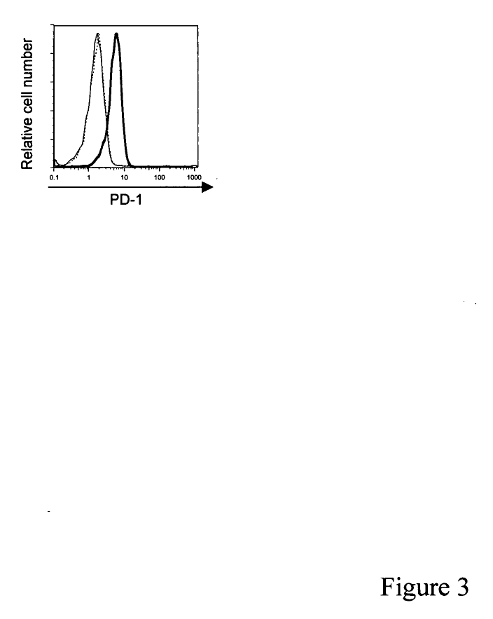

Since G8 γδ T cells recognize oligodendrocyte precursor/oligodendrocytes in vitro, we asked whether their accumulation in CNS can be induced and whether this would correlate with the induction of CNS pathology. It is well documented that EAE induction in susceptible strains of mice requires the injection of myelin components together with CFA containing heat-killed Mycobacterium tuberculosis. There were reports showing that γδ T cells can be activated in Mycobacterium tuberculosis infected animals. We therefore injected G8 transgenic mice, which are on the EAE-resistant BALB/c background, with CFA containing heat-killed Mycobacterium tuberculosis but without any myelin components. We found that on day 6, G8 γδ T cells in the peripheral blood were found to express the activation markers CD69 and PD-1. On day 11, almost the entire G8 γδ T cell population in the peripheral blood were PD-1-positive (FIG. 3B). No induction of G8 cell activation was found in incomplete Freund's adjuvant (IFA) (without heat-killed Mycobacterium tuberculosis) injected G8 mice, or in CFA injected BALB/c mice (FIG. 3B).

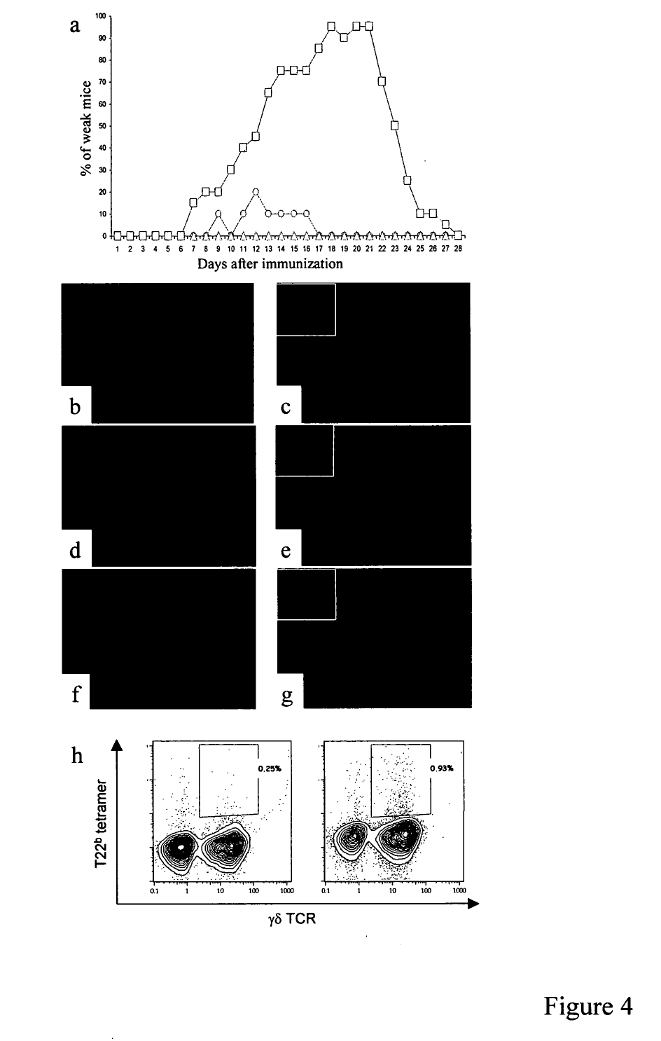

Parallel with these observations, we also found that as early as day 7 after CFA injection, some of the G8 mice show a weakness of the limbs (scored as 2 and 3 in clinical evaluation of EAE). On day 12, around 40 to 50% of mice showed symptoms. On day 16, all G8 mice injected with CFA showed weakness of the limbs. No mice showed paralysis and all mice recovered four weeks after the injection (FIG. 4a). Immunohistochemical analysis of the spinal cord sections of CFA injected G8 mice showed the presence of γδ T cells (GL-3 positive) and increased numbers of macrophages and microglia (Mac-1, F4/80 positive) (FIG. 4). However, despite the fact that G8 cells induced rapid demyelination in in vitro myelinating cultures, no obvious demyelination has been observed in the spinal cords of these mice. This is not un-expected. Since all G8 mice recovered from clinical symptoms, it is likely that if there is an initial demyelination, it must be mild and can be repaired readily by OPCs and OLs in vivo. Consistent with the lack of induction of G8 cell activation in IFA injected G8 mice, no clinical symptoms, or the accumulation of infiltrates were observed in these animals.

With a T22-tetrameric staining reagent, we further demonstrated that CFA injection induced the expansion/activation of the T10/T22 specific γδ T cell population in normal BALB/c mice (FIG. 3C). Taken together, these results indicate that CFA induces the activation of T10/T22 specific γδ T cells. It is likely that the recognition of T10/T22 expressed on myelin forming cells leads to the accumulation of these cells in the CNS. Our preliminary results showed that chemokines and macrophage activating growth factors were found in the co-culture of γδ T cells stimulated with myelin forming cells. Therefore, the γδ T cell infiltration may lead to the activation and accumulation of macrophages/microglia to initiate the immunological pathology in the CNS.

These results are consistent with the observation that a disproportionally high percentage of γδ T cells (up to 30% of the total number of T cells) are associated with acute MS plaques (Wucherpfennig et al. (1992) Proc. Natl. Acad. Sci. USA 89, 4588-4592). Further, mice depleted of γδ T cells exhibit decreased chemokine responses at the onset of EAE. More significantly, EAE induction in mice with a disrupted TCRδ chain gene (TCRδ^{-/-} mice) showed no cellular infiltration in the spinal cord and decreased immune responses and disease severity.

Similar to T10/T22 in the mouse, activation/infection/stress induced self antigens—MICA/B, CD1 and small phosphate containing compounds have been described as antigens of the human γδ T cells. Although it is not clear whether these and other human γδ T cell ligands are also expressed on neuronal cells, our results presented here suggest that γδ T cells have an important role in initiating inflammatory demyelinating diseases by directly recognizing self antigens expressed on cells of the CNS. Disrupting the interaction between γδ TCR with its ligand or depleting γδ T cells in patients may provide new therapeutic strategies for MS.

Methods

RT-PCR reactions. 2 μg of total RNA were reverse transcribed (RT) and 10% of RT products was used for each reaction. RNA samples, which were prepared from different mouse tissues, cells and cocultures, were reverse transcribed and subjected to 20 cycles PCR with specific primers for T10 (SEQ ID NO:8) (GCCATCTCAGGGTGAGGG and (SEQ ID NO:9) ATGGGTTCACACTCGCTTAGG); for β-actin (SEQ ID NO:10) (TGGGCCGCTCTAGGCACC and CTCTTTGATGTCACGCACG).

Purification and culture of OPCs and oligodendrocytes. To purify OPCs from BALB/c mice, whole brain was obtained from a P9 mouse and OPCs were purified by sequential immunopanning. Briefly, brain cell suspensions were prepared by dissociated the brain in papain buffer (20 units/ml, Worthington) for 75 min at 37° C. and then placed the cells on the anti-Thy1.2 plate for 30 min at room temperature. The nonadherent cells were transferred to the anti-GC plate to remove oligodendrocytes. Finally, OPCs were collected from the 04 dish. Purified OPCs were cultured on Chambered Coverglass (4.2 cm², Nalge Nunc International) that had been coated with Poly D-Lysine (10 μg/ml). The serum-free medium, DMEM (GIBCO), included bovine serum albumin (BSA, 4%), transferrin (100 μg/ml), progesterone (60 ng/ml), putrescine (16 μg/ml), sodium selenite (40 ng/ml), N-acetyl cysteine (5 μg/ml), sodium pyruvate (1 mM), insulin (5 μg/ml), glutamine (2 mM), CNTF (1 ng/ml), PDGF (10 ng/ml) and NT-3 (1 ng/ml). For in vitro differentiation of oligodendrocytes, triiodo-thyronine (40 ng/ml) was additionally added into the medium but PDGF and NT-3 were omitted from the medium.

Cell cultures. For all γδ T cell blasts, naïve γδ T cells were prepared by labeling splenocytes with GL-3-FITC (BD PharMingen) and isolating FITC-positive cells based on their binding to anti-FITC MicroBeads (Miltenyi Biotec Inc.) and then purified by MS column (Miltenyi Biotec Inc.). γδ T cell blasts were generated by stimulating purified splenic γδ T cells in plates pre-coated with GL-4 (1 μg/ml) and anti-CD28 (0.5 μg/ml) and with rIL-2 after 24 hours of stimulation. After 7 days, γδ T cells were purified again before using. Myelinating dissociated DRG explants were prepared as previously described. Coverslips were placed onto a siliconized glass surface and 1×10⁶ γδ T cells were added onto the myelinated explants. Blocking of demyelination with T22/β2M monomer was performed with 150-200 □g/ml unlabeled monomer that was preincubated with 1×10⁶ G8 cells for 45 min at RT before adding them into myelinating cocultures and the monomer was present during the 24 hr incubation. The structure of the myelin was analyzed 24 hours later by detecting MBP with immunocytochemistry (Chemicon).

Mice and injection. BALB/c mice were obtained from Stanford Department of Comparative Medicine colonies. G8 mice were the kind gift of Dr. Stephen M. Hedrick. G8/Rag^{-/-} mice were generated by crossing G8 male mice to Rag-2^{-/-} female mice, and screened by PCR. Rag-2^{-/-} mice were obtained from Taconic. BALB/c and G8 mice aged 5-8 wk received 100 μl of CFA containing 400 μg of heat-killed M. tuberculosis H37Ra (Difco Laboratories) on day 0. For the controls, G8 mice were injected with IFA without M. tuberculosis.

Immunohistochemistry. Half of brain and spinal cord from the same mice were collected for frozen sections. Tissues were embedded in Tissue-Tek O.C.T. Compound (VWR Scientific Products) quick-frozen in dry ice mixed with isopentane and sections (5 μm) were fixed by 4% (w/v) paraformaldehyde for 15 min, blocked with 2% BSA/5% normal hamster serum/5% normal mouse serum/1:100 Fc block (CD16/CD32, BD PharMingen) for 1.75 hours, pre-treated with 2% H₂O₂ in PBS for 20 min, and stained with GL-3-FITC (1:100, BD PharMingen) or Mac-1-FITC, F4/80-FITC (M1/70.15, CI:A3-1; 1:100, CALTAG Lab.) for 2 hours and then counterstained with anti-fluorescein-POD (1:100, Boehringer Mannheim) for 1 hour. Slides were developed using Tyramine-fleorescein (1:1000) in 200 mM Tris pH8.8/10 mM Imidazole.

Flow cytometry. The tetrameric staining of splenic γδ T cells was carried out as previously described. Briefly, the total spleen cells (1×10⁸/ml) were blocked with 10% normal mouse serum/10% normal hamster serum/1:100 Fc block/1:100 anti-TNP-Cy-Chrome (A19-3, BD PharMingen)/1:400 CD19-Cy-Chrome (6D5, southern Biotechnology associates) in 0.5% BSA/2 mM EDTA/PBS for 1 hour, stained with the FITC conjugated GL-3 antibody for 1 hour, and enriched the γδ T cells by anti-FITC MicroBeads (Miltenyi Biotec Inc.) as described in cell cultures. The purified γδ T cells were then incubated with PE-conjugated T22 tetramer (12 μg/ml) for 1.5 hours on ice. Propidium iodide (PI) at 1 μg/ml was added after the final wash. The blood from immunized mice were collected every 2 days, and performed a series staining monitoring the αβ T cells, B cells, macrophages and γδ T cells. Anti-CD69 (H1.2F3; 1:100, BD PharMingen) and anti-PD-1 (J43; 1:100, eBioScience) were used as the activation marker for γδ T cells. PI-positive cells and Cy-Chrome-positive cells were excluded from the analysis.

### Example 2

### Shakie

Mice expressing the T cell receptors of the G8 γδ T cell clone, which is specific for the non-classical MHC class I molecules T10/T22, have been generated to study the development of γδ T cells. Surprisingly, we found that some of the G8 transgenic mice maintained in our mouse colony spontaneously develop a novel mutation in the Mbp gene. These mice show marked tremors of the hindquarters at weaning, termed G8/Shakie. In this study, we crossed out the shakie (sha) mutation from G8 transgenic background, and the characterization of Shakie provides an excellent model for the studies of the MBP biology. In addition to Shakie, the G8/Shakie model further allowed us to investigate the relationship between γδ T-cells and neurological autoimmune diseases such as MS.

Methods

Mice. G8 TCR transgenic mice were generated from the γδ T cell clone G8. These transgenic mice have been backcrossed onto the BALB/c (H-2d) background since 1989 and maintained as homozygous with respect to the transgenes for at least the past six years at Stanford. Shakie mutant was discovered in G8 transgenic mice colony and has been bred out from transgenic background. G8/Shakie and Shakie are maintained as heterozygous.

Genomic Southern Blot and Northern Blot. 3 μg of genomic DNA were digested with SacI, XmnI and PstI for the genomic Southern blot analysis. For the northern blot analysis, 7.5 μg of total RNA from brains of G8/Shakie and its littermates were electrophoreses in 0.8% agarose gel and transferred onto Hybond N+ membrane (Amersham). Probes were generated by RT-PCR amplification with the following primers: for Southern blot, (SEQ ID NO:1) 5′-TCACACACGAGMC-TACCCATT-3′; (SEQ ID NO:2) 5′-GCTCCACGGGATTMGAGAG-3′; for northern blot, (SEQ ID NO:3) 5′-ATCTGCTGAGAAGGCCAGTAAG-3′; (SEQ ID NO:4) 5′-TGGGTAGTTCTCG-TGTGTGAGTC-3′.

Rapid amplification of 3′ cDNA ends (RACE) PCR. 5 μg of total RNA from the brains of G8/Shakie mice were used for reverse transcription (RT) with (dT)₁₅-T7 promoter oligo: (SEQ ID NO:5) 5′-AAACGACGGCCAGTGAATTGTMTACGACTCACTATAGGCGCTTTTTTTTTTTTTTT-3′. 10% of the RT products were subjected to 20 cycles of PCR amplification with specific primers: (SEQ ID NO:6) 5′-CCCTCACAGCGATCCMGTA-3′; (SEQ ID NO:7) 5′-AAACGACGGCCAGTGAATTGTMTAC-GACTCACTATAGGCGC-3′. The PCR product was cloned using TOPO TA Cloning Kit (Invitrogen) and sequenced.

Western Blot Total brain proteins were extracted with modified RIPA buffer (50 mM Tris base, 150 mM NaCl, 1% NP40, 0.25% Na Deoxycholate, 1 mM EDTA). The expression of MBP in BALB/c/G8^{tg}-Mbp^{sha+}, G8/Shakie and Shakie was revealed by 12.5% SDS page and visualized by anti-MBP mAb (MAB387, Chemicon international).

Immunohistochemistry. The brain and spinal cord from the same mice were collected and embedded in Tissue-Tek O.C.T. Compound (VWR Scientific Products) quick-frozen in dry ice mixed with isopentane. Tissue sections (5 μm) were fixed with 4% (w/v) paraformaldehyde (PFA) for 15 min, blocked with 50% goat serum/0.1% NP-40/PBS for 1 hour, stained with anti-NG2 (CHEMICON International) overnight at 4° C. and counterstained with anti-rabbit-Alexa 488 for 1 hour. For CC1 staining, sections were blocked with 10% goat serum/10% horse serum/1% BSA/0.3% Triton X-100 for 2 hours, stained with CC1 (Oncogene.) overnight and 1 hour counterstained with anti-mouse-Alexa 488. For macrophages and γδ T-cells staining, sections were blocked with 2% BSA/5% normal hamster serum/5% normal mouse serum/1:100 Fc block (CD16/CD32, BD PharMingen) for 1.75 hours, pre-treated with 2% H₂O₂ in PBS for 20 min, and stained with GL-3-FITC (1:100, BD PharMingen) or Mac-1-FITC (M1/70.15; 1:100, CALTAG Lab.) for 2 hours and then counterstained with anti-fluorescein-POD (1:100, Boehringer Mannheim) for 1 hour. Slides were developed using Tyramine-fluorescein (1:1000) in 200 mM Tris pH8.8/10 mM Imidazole.

Results

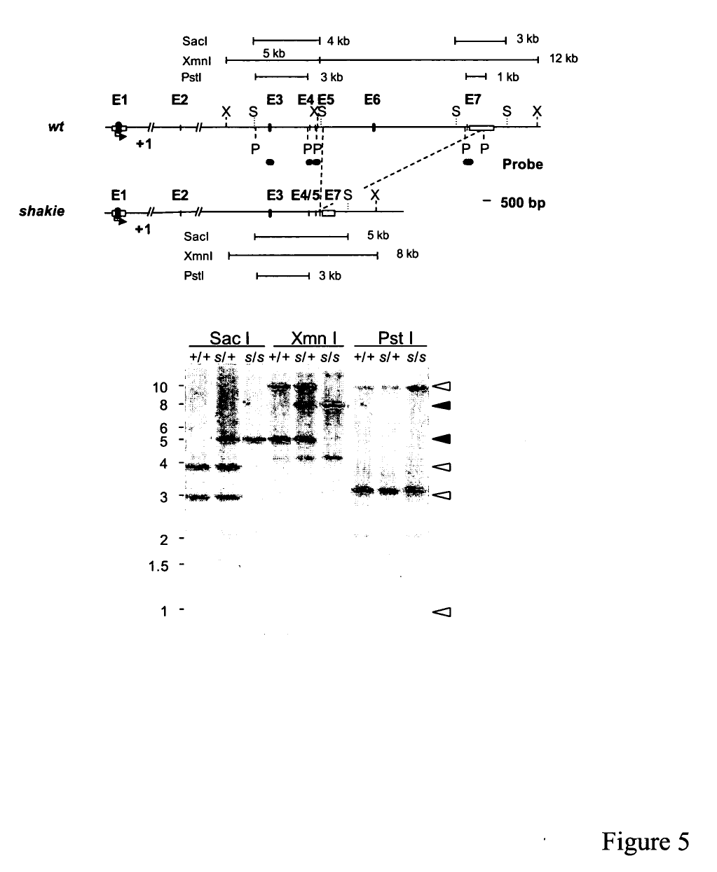

G8/Shakie carries a novel MBP mutation. G8/Shakie is a spontaneous mouse mutant derived from BALB/c mice expressing a transgenic G8 γδ TCR; it displays marked tremors of the hindquarters at two weeks, hence its name, and has an average lifespan of 70-80 days. Preliminary observation that 20-25% of certain crosses shows a G8/Shakie phenotype suggested a single locus recessive genetic defect. The similarity of this phenotype with Shirever (shi/shi) mice, which have an autosomal recessive mutation of the Mbp gene (Molineaux et al., 1986), lead us to question the integrity of G8/Shakie's Mbp gene. Genomic Southern blot analysis revealed a 9 kb deletion, which removes exon 6 and the translated part of the terminal exon 7 in G8/Shakie's Mbp gene (FIG. 5).

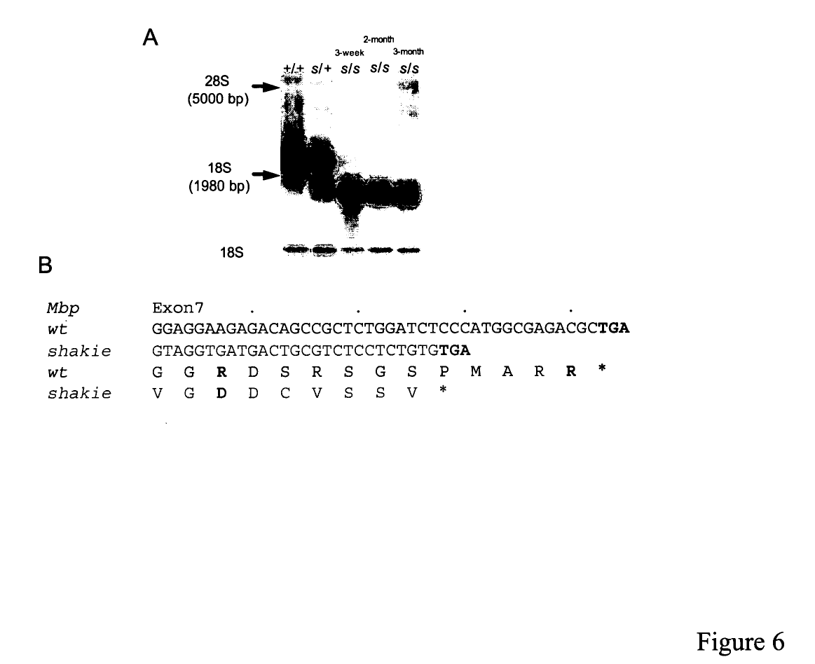

The expression of truncated MBP in Shakie mutant. The deletion in G8/Shakie's Mbp gene is not as severe as the one observed in Shirever mice that removes the last four exons. However, the main difference between the two mice is that contrarily to Shirever, the partially deleted Mbp gene of G8/Shakie can be transcribed as indicated by northern blot (FIG. 6A). The corresponding mRNA was characterized by RT-PCR, 3′ RACE-PCR amplification and sequencing. One Mbp mRNA was fully characterized; it is similar to the mRNA encoding the 17.2 kDa isoform of MBP (lacking exon 6), but has a new 3′ end, which use 27 bp of the untranslated part of terminal exon 7 (FIG. 6B). The usual 14 amino acids encoded by exon 7 and found in all the isoforms of MBP are replaced in Shakie by 9 new amino acids. Among the amino acids replaced are two out of the five arginine residues that are important for the attachment of MBP to the myelin membrane. In Shakie's MBP, one positively charged arginine is replaced by a negatively charged aspartic acid and the second arginine is absent (FIG. 6B). This change mimics the phenomenon of arginine citrullination observed in MBP, which convert the positively charged arginine residues to uncharged citrulline (Cit), lead to a reduction in the surface charge and could be operative in demyelinating diseases such as chronical MS, and fulminating MS (marburg's disease).

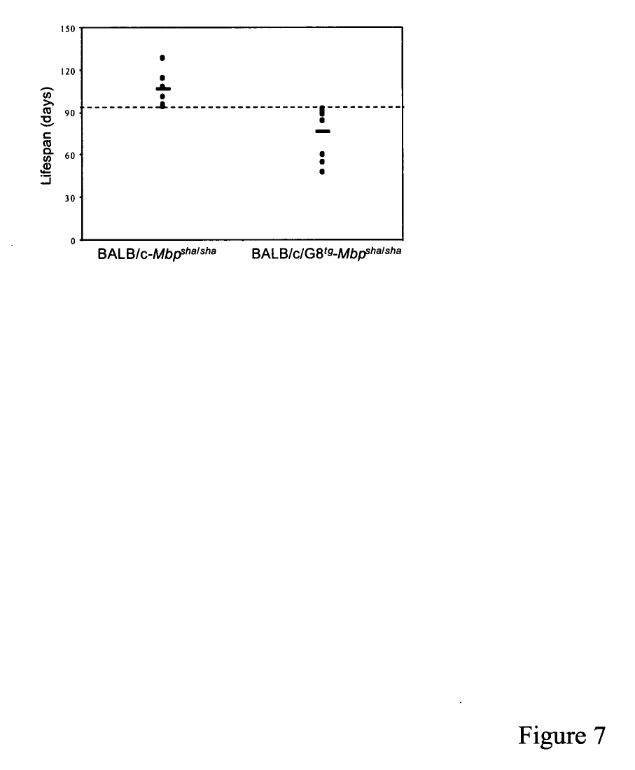

The phenotype of Shakie mutant. To better understand the effect of the high number of total γδ T-cells in G8 mice (˜40% splenic γδ T-cells versus ˜0.4% in wild type) on the G8/Shakie phenotype (BALB/c/G8^{tg}-Mbp^{sha/sha}), we crossed out this mutant (sha) to remove the G8 transgenic background while keeping the sha character. The resulting mice (BALB/c-Mbp^{sha/sha}) display a shaking phenotype similar to that observed with G8/Shakie. However, the BALB/c-Mbp^{sha/sha}, called Shakie, have an average lifespan of 107 days, which constitutes a statistically significant increase over the 76 days average lifespan of G8/Shakie (p<0.01) (FIG. 7). The maximum lifespan is also extended in Shakie versus G8/Shakie. Thus, the presence of an increased number of γδ T-cells bearing the G8 receptor (specific to T10/T22) significantly reduces the lifespan of G8/Shakie.

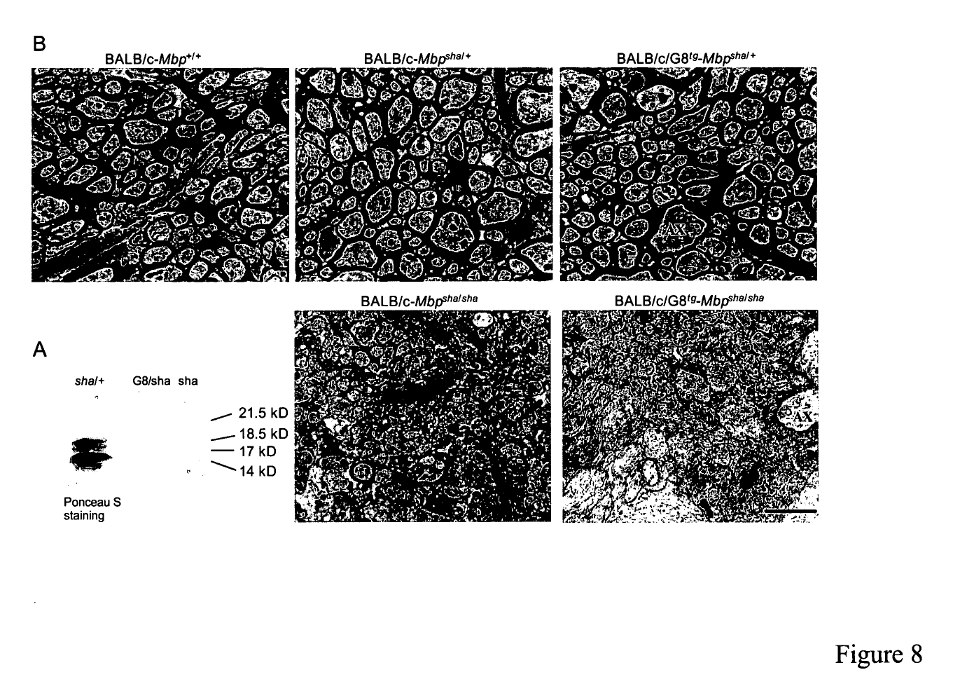

Myelin deficiency in Shakie mice. To investigate the factor responsible of the decrease in lifespan in G8/Shakie, we analyzed the MBP protein expression in the brain by Western blot. Using standard quantities of total proteins (5 μg), it is not possible to reveal the presence of the protein in both G8/Shakie and Shakie, but as the quantity of total protein used is increased to 100 pg, two MBP isoforms can be detected in Shakie but not in G8/Shakie (FIG. 8A). Consistent with this observation, Shakie has more visible myelin than G8/Shakie as illustrated by electron micrographs (EM) of the optic nerve (FIG. 8b). While both mice show a significant decreased myelin when compared to the normal level of the heterozygous mutant (sha/+) or the wild type (FIG. 8B), the intensity of this decrease is quite different, as almost all the axons of the optic nerve are naked in G8/Shakie.

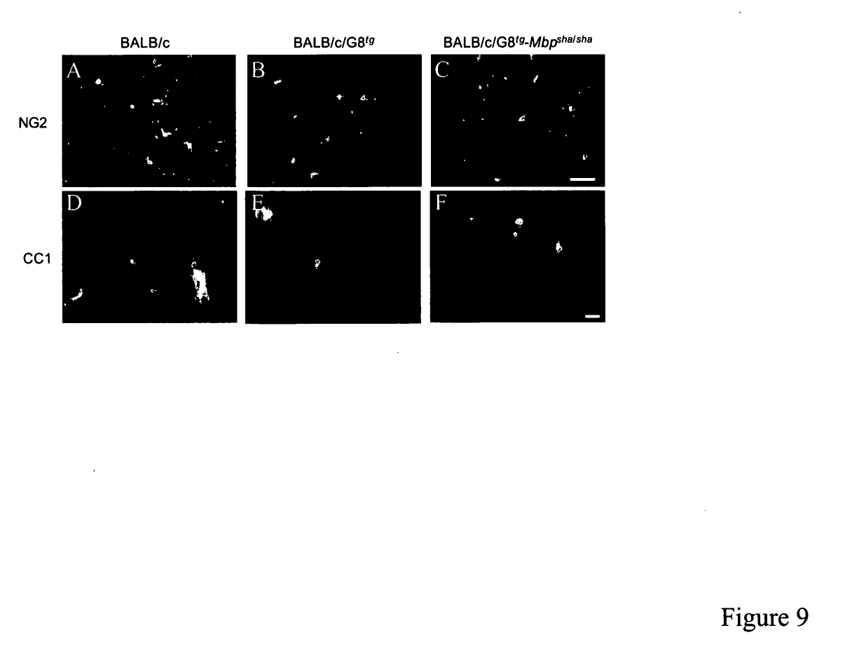

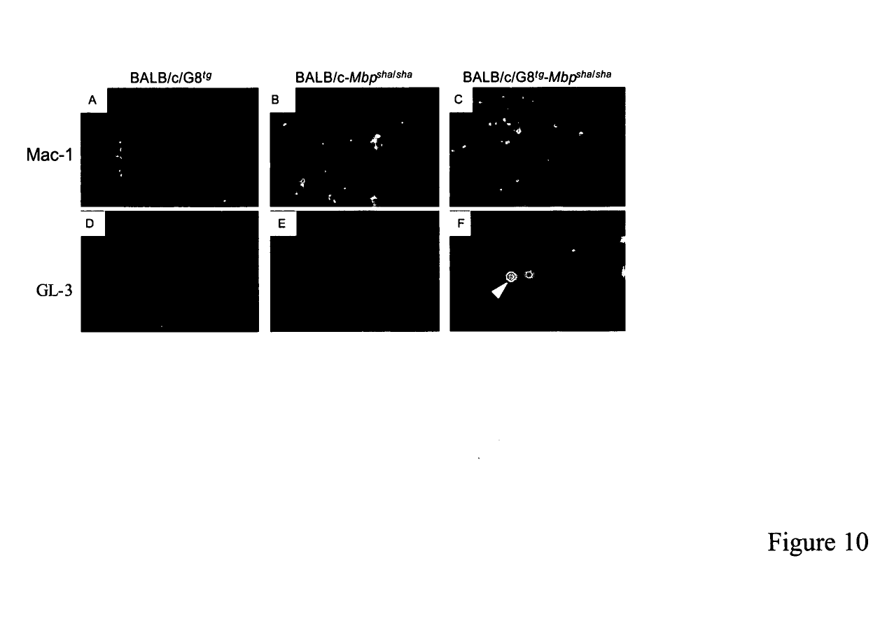

CNS inflammation and γδ T cells infiltration. To understand why G8/shakie shows a nearly complete absence of myelin, we studied the development of the CNS myelin forming cells, oligodendrocytes, and oligodendrocyte precursor cells (OPCs) in G8/Shakie, G8 and BALB/c mice. As shown in FIG. 9, despite the absence of myelinated axons in the surrounding area, the numbers and appearance of OPCs (NG2-positive) and oligodendrocytes (CC1-positive) in the white matter area of the G8/Shakie's brain are normal. Thus, an interruption of OPC development is unlikely to be the cause of complete myelin deficiency in G8/Shakie. In addition to the lack of myelin, signs of inflammation are observed in the CNS of Shakie and G8/Shakie. As shown in FIG. 10, macrophage/microglia (Mac-1-positive) are increased in the white matter region of the spinal cord in both Shakie and G8/Shakie (FIG. 10B,C). In contrast, a massive accumulation of γδ T-cells (GL-3-positive) is only found in G8/Shakie (FIG. 10F). This indicates that the infiltration of T10/T22 specific G8 γδ T-cells in the white matter may be responsible for the different myelin amount between Shakie and G8/Shakie.

MS is a complex disease, which involves environmental, genetic and immunological factors and ultimately leads to the axon demyelination. The questions of the relative importance of each of these factors and how exactly the pathology is initiated have always been difficult to answer. G8/Shakie provides a perfect background to tackle these questions.

Shakie's neurological disorder differs from that previously described for Shiverer mice in a number of significant ways, including the expression of two MBP isoforms and macrophage/microglia accumulation in the white matter tracts in Shakie but not in Shiverer mice. Furthermore, overexpression of the 17.2 kDa MBP isoform can restore the myelination in Shiverer mice, but while Shakie can also express the 17.2 kDa isoform, it displays a strong myelin deficiency. The presence of a new terminal sequence in Shakie's MBP isoforms could be responsible for this difference, as two out of the five arginine residues (positive charge) important for the attachment of MBP to the myelin membrane are absent in Shakie's MBP. These mutations in Shakie mimic arginine citrullination (conversion of positively charged Arginine to uncharged Citrulline (Cit)), a phenomenon that was shown to have direct consequences for MBP, such as a reduced ability to aggregate lipid vesicles, a slightly greater susceptibility to digestion by Cathepsin D, a greater proportion of random secondary structures, and different conformational responses to lipid. This reduction in cationicity is also though to play an important role in the pathogenesis of demyelinating diseases, as indicated by the high incidence of citrullinated MBP in chronic (45%) and fulminating (90%) MS patients as compared to healthy individuals (20%).

Because of their backgrounds, where MBP is naturally modified to predispose to diseases like MS, Shakie and G8/Shakie constitute good models to study the progression of the immune responses associated with the disease. In addition to the deficiency of myelin, an increased number of macrophage/microglia in the CNS was also observed in both mice. The reason for this observation is not clear, but it is possible that the enhanced number of Cathepsin D digested MBP debris following natural deamination could attract macrophage/microglia and lead to their accumulation in the CNS, and then up regulates the activation/infection induced self-antigens such as T10 in the microenvironment. While this does not seem to induce further visible immune responses in Shakie, the consequence of this is revealed in G8/Shakie, which clearly harbors signs of γδ T-cells-mediated pathogenesis that result in complete myelin deficiency as suggested by the massive accumulation of γδ T-cells in the white matter regions of CNS.

The complete amino acid sequence of MBP was determined in 1971. The biological function of different isoforms of MBP is continually discovered. Recently, MBP-related transcripts were found not only present in the nervous system, but also in bone marrow and immune system. The most abundant MBP-related mRNAs and proteins, called hematopoietic MBP (HMBP), are present in more than 95% of thymic T cells, as do mature T cells, B cells and all types of myeloid lineage cells (Marty et al., 2002). The carboxyl terminal domain of MBP is present in all classical MBP isoforms and most HMBP isoforms. Therefore, the inherent truncated shakie MBP provides an opportunity to investigate in detail the function of this terminal domain both in the myelin biology of the nervous system and in the immune responses of the entire hematopoietic system.

1. A method of treating an inflammatory demyelinating disease, the method comprising:
administering to a patient an effective dose of an agent the interferes with the binding between a γδ T cell receptor and ligand.

2. The method according to claim 1, wherein said ligand is an MHC molecule.

3. The method according to claim 1, wherein said MHC molecule is a class Ib MHC or class II MHC.

4. The method according to claim 3, wherein said Class Ib MHC is encoded in the T region, or a homolog thereof.

5. The method according to claim 4, wherein said Class Ib MHC is a T10, T22 or functional counterpart.

6. The method according to claim 1, wherein said administering prevents initiation of said disease.

7. The method according to claim 1, wherein said inflammatory demyelinating disease is multiple sclerosis.

8. A method of diagnosing or staging an inflammatory demyelinating disease, the method comprising:
detecting increased numbers of γδ T cells in the central nervous system of an individual.

9. The method according to claim 8, wherein said detecting comprising in vivo imaging.

10. The method according to claim 8, wherein said detecting comprises in vitro analysis of a sample from said individual.

11. The method according to claim 8, wherein said γδ T cells specifically interact with a Class Ib MHC molecule.

12. The method according to claim 11, wherein said Class Ib MHC molecule is expressed on cells of the oligodendrocyte lineage.

13. The method according to claim 12, wherein said Class Ib MHC is encoded in the T region, or a homolog thereof.

14. The method according to claim 13, wherein said Class Ib MHC is a T10, T22 or homolog protein.

15. The method according to claim 12, wherein increased numbers of said γδ T cells is indicative of a predisposition to, or initiation of, said inflammatory demyelinating disease.

16. A method for identifying a therapeutic agent for treatment of inflammatory demyelinating disease, the method comprising:
detecting the ability of an agent to inhibit the interaction between a γδ T cell receptor and ligand, thereby identifying an inhibitor that is useful as a therapeutic agent,
wherein inhibition of said interaction is therapeutic in the treatment of inflammatory demyelinating disease.

17. The method according to claim 16, wherein said ligand is a Class Ib MHC molecule.

18. The method according to claim 17, wherein said Class Ib MHC is encoded in the T region, or a homolog thereof.

19. The method according to claim 18, wherein said Class Ib MHC is a T10, T22 or homolog protein.

20. An animal model for inflammatory demyelinating disease, said model comprising:
a non-human mammal comprising a transgene encoding at least one chain of a γδ T cell receptor;
wherein T cells in said mammal are activated as a result of administration of an adjuvant.

21. The model according to claim 18, further comprising a mutation in myelin basic protein that has reduced cationicity in residues utilized for the attachment of MBP to the myelin membrane.

22. The model according to claim 20, wherein said γδ T cell receptor recognizes a class Ib MHC molecule.

23. The method according to claim 22, wherein said Class Ib MHC is encoded in the T region, or a homolog thereof.

24. The method according to claim 23, wherein said Class Ib MHC is a T10, T22 or homolog protein.

25. The model according to claim 20, wherein said adjuvant comprises bacterial antigens.


SEQUENCE LISTING (10 sequences)

SEQ ID NO: 1 - DNA, 22 bp, Mus musculus
tcacacacgagaactacccatt

SEQ ID NO: 2 - DNA, 20 bp, Mus musculus
gctccacgggattaagagag

SEQ ID NO: 3 - DNA, 22 bp, Mus musculus
atctgctgagaaggccagtaag

SEQ ID NO: 4 - DNA, 23 bp, Mus musculus
tgggtagttctcgtgtgtgagtc

SEQ ID NO: 5 - DNA, 57 bp, Mus musculus
aaacgacggccagtgaattgtaatacgactcactataggcgcttttttttttttttt

SEQ ID NO: 6 - DNA, 20 bp, Mus musculus
ccctcacagcgatccaagta

SEQ ID NO: 7 - DNA, 42 bp, Mus musculus
aaacgacggccagtgaattgtaatacgactcactataggcgc

SEQ ID NO: 8 - DNA, 18 bp, Mus musculus
gccatctcagggtgaggg

SEQ ID NO: 9 - DNA, 21 bp, Mus musculus
atgggttcacactcgcttagg

SEQ ID NO: 10 - DNA, 37 bp, Mus musculus
tgggccgctctaggcaccctctttgatgtcacgcacg